(19) United States
(12) Patent Application Publication
(10) Pub. No.: US 2024/0324922 A1
(43) Pub. Date: Oct. 3, 2024

(54) SYSTEM FOR DETECTING HEALTH EXPERIENCE FROM EYE MOVEMENT

(71) Applicant: Worcester Polytechnic Institute, Worcester, MA (US)

(72) Inventors: Soussan Djamasbi, Natick, MA (US); Javad Norouzi Nia, Dewitt, MI (US); Doaa Alrefaei, Northborough, MA (US); Diane Strong, Worcester, MA (US); Randy Paffenroth, Northborough, MA (US)

(73) Assignee: Worcester Polytechnic Institute, Worcester, MA (US)

(21) Appl. No.: 18/584,731

(22) Filed: Feb. 22, 2024

Related U.S. Application Data
(60) Provisional application No. 63/448,154, filed on Feb. 24, 2023.

(51) Int. Cl. A61B 5/16; A61B 3/113; A61B 5/00

(52) U.S. Cl. CPC A61B 5/163; A61B 3/113; A61B 5/4824; A61B 5/7282

(57) ABSTRACT

A health experience detection system comprises an eye-tracking device and a health experience detection device disposed in electrical communication with the eye-tracking device. The health experience detection device comprises a controller having a memory and a processor. The controller is configured to: receive eye-movement data from the eye-tracking device, the eye-movement data associated with eye-movement of a user and comprising at least one of saccade event data and fixation event data, apply the eye-movement data to a health experience identification engine to generate a health experience identifier associated with the user, and output a notification regarding the health experience identifier of the user as associated with the eye-movement data.

### RELATED APPLICATIONS

This patent application claims the benefit of U.S. Provisional Application No. 63/448,154, filed on Feb. 24, 2023, entitled “System for Detecting Health Experience From Eye Movement,” the contents and teachings of which are hereby incorporated by reference in their entirety.

### BACKGROUND

Health professionals can use a person's self-assessment of their health experience to determine how, or if, to treat the patient. For example, one such health experience is chronic pain which is defined as pain that persists for at least three months. In the United States, chronic pain is one of the most commonly experienced chronic conditions, afflicting about 50 million (i.e., 1 out of 5) adults and, as such, is a major public health problem.

Treatment of a health experience, such as chronic pain, can start by assessing how intensely the experience is experienced by a person and how severely it interrupts the person's daily activities. Typically, chronic pain is assessed based upon the results of a survey provided to the person. The survey allows the person to provide ratings that capture the person's level of pain intensity, as well as the degree to which pain interferes with their physical and day-to-day activities. Based on the results of the survey, the health care professional can determine if the person's pain is indeed chronic pain or, instead, should be considered as acute pain. With this determination, the healthcare professional can develop a treatment plan for the person to address the health experience.

### SUMMARY

Conventional assessment of a person's health experience suffers from a variety of deficiencies. As provided above, a person's health experience, such as chronic pain, can be provided via a survey that allows a person to report self-assessed ratings. However, because self-reported pain measures require individuals to report a complex multifaceted phenomenon as a single score, they can only reveal a narrow view of the subjective pain experience. Additionally, despite providing an opportunity for individuals to convey their pain experience, self-reported measures lack the objectivity that is needed to make significant improvements in chronic pain treatment and research.

By contrast to conventional assessment techniques, embodiments of the present innovation relate to a system for detecting health experience from eye movement. For example, those experiencing pain differ in their allocation of attention to pain stimuli and they differ in their cognitive processes such as those involved in decision making. These results, suggest that pain affects how people process information and how they use that information to make decisions. Because eye-tracking provides unobtrusive insights into attention and cognition related to decision making, in one arrangement, the health experience identification system is configured to differentiate the health experiences of people, such as between those who are experiencing chronic pain and those who are pain free, and thus provide biomarkers of pain. Further, the health experience identification system can be configured to identify a variety of health experiences, such as anxiety.

Embodiments of the innovation relate to a health experience detection system configured to track a user's eye movements and to predict a health experience associated with the user via a health experience identification engine. For example, a health experience detection device associated with the system can apply the user's eye movements data to a health experience identification engine in order to accurately and objectively predict the user's health experience via the eye-movement data. By providing an objective measure of the user's health experience (e.g., chronic vs. acute pain, chronic vs. acute anxiety, etc.), the health experience detection device can provide a health care professional with the information needed to accurately assess and treat the user's health condition.

In one arrangement, the innovation relates to a health experience detection system comprises an eye-tracking device and a health experience detection device disposed in electrical communication with the eye-tracking device. The health experience detection device comprising a controller having a memory and a processor, the controller configured to: receive eye-movement data from the eye-tracking device, the eye-movement data associated with eye-movement of a user and comprising at least one of saccade event data and fixation event data, apply the eye-movement data to a health experience identification engine to generate a health experience identifier associated with the user, and output a notification regarding the health experience identifier of the user as associated with the eye-movement data.

In one arrangement, the innovation relates to, in a health experience detection device, a method for providing a health experience identifier of a user. The method comprises receiving, by the health experience detection device, eye-movement data from an eye-tracking device, the eye-movement data associated with eye-movement of the user and comprising at least one of saccade event data and fixation event data; applying, by the health experience detection device, the eye-movement data to a health experience identification engine to generate a health experience identifier associated with the user; and outputting, by the health experience detection device, a notification regarding the health experience identifier of the user as associated with the eye-movement data.

In one arrangement, the innovation relates to a health experience detection device, comprising a controller having a memory and a processor. The controller is configured to: receive eye-movement data from an eye-tracking device, the eye-movement data associated with eye-movement of a user and comprising at least one of saccade event data and fixation event data; apply the eye-movement data to a health experience identification engine to generate a health experience identifier associated with the user; and output a notification regarding the health experience identifier of the user as associated with the eye-movement data.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 illustrates a schematic diagram of a health experience identification system, according to one arrangement.

FIG. 2 illustrates a flow chart of a procedure performed by a health experience detection device of the health experience identification system of FIG. 1, according to one arrangement.

FIG. 3 illustrates a schematic diagram of the health experience detection device of FIG. 1 configured to generate a pain identifier and objective pain level data associated with eye movement data, according to one arrangement.

FIG. 4 illustrates a schematic diagram of the health experience detection device of FIG. 1 configured to generate a mental health identifier and objective mental health level data associated with eye movement data, according to one arrangement.

FIG. 5 illustrates a schematic diagram of a health experience identification system configured to transmit a visual stimuli sample to a display, according to one arrangement.

FIG. 6 illustrates a schematic diagram of the health experience detection device of FIG. 1 configured to generate a user-specific health experience identification engine, according to one arrangement.

### DETAILED DESCRIPTION

Embodiments of the innovation relate to a health experience detection system configured to track a user's eye movements and to predict a health experience associated with the user via a health experience identification engine. For example, a health experience detection device associated with the system can apply the user's eye movements data to a health experience identification engine in order to accurately and objectively predict the user's health experience via the eye-movement data. By providing an objective measure of the user's health experience (e.g., chronic vs. acute pain, chronic vs. acute anxiety, etc.), the health experience detection device can provide a health care professional with the information needed to accurately assess and treat the user's health condition.

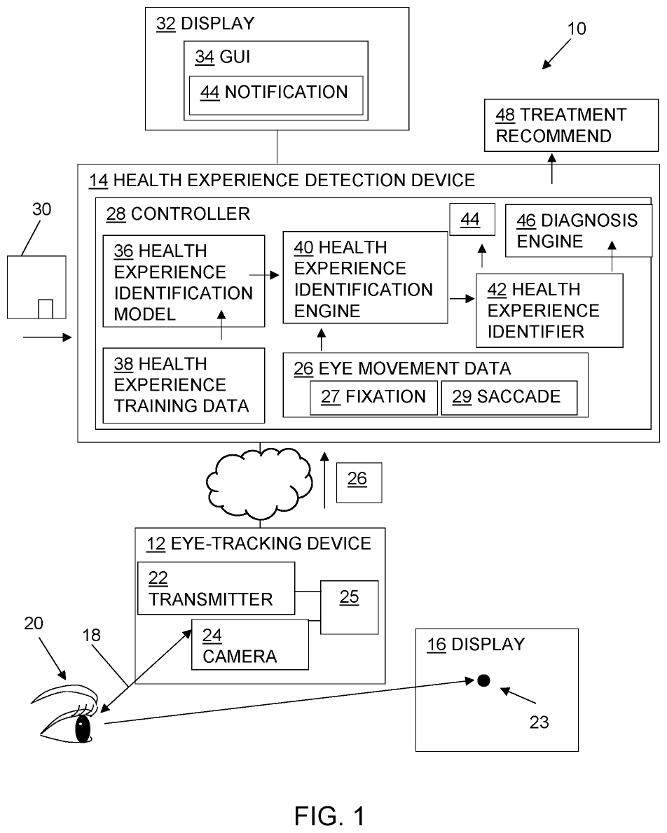

FIG. 1 illustrates a schematic representation of a health experience identification system 10, according to one arrangement. As illustrated, the health experience identification system 10 includes an eye-tracking device 12 disposed in electrical communication with a health experience identification device 14. In one arrangement, each of the eye-tracking device 12 and the health experience identification device 14 are configured as standalone devices disposed in electrical communication with each other via a network 27, such as a local area network (LAN) or a wide area network (WAN). In one arrangement, the health experience identification system 10 includes both the eye-tracking device 12 and the health experience identification device 14 as part of a single device.

The eye-tracking device 12 is configured to detect the position of a user's eye relative to a field of view, such as a display 16 or any image received by the user, whether generated electronically or otherwise, based upon the measured position of the user's eye in space. For example, the eye-tracking device 12 can include an infra-red (IR) transmitter 22 and camera 24 disposed in electrical communication with a controller 25, such as a processor and a memory. The transmitter 22 is configured to direct a light 18, such as an infrared (IR) light, against a user's eye 20. The light 18 allows the camera 24 of the eye-tracking device 12 to identify the pupil of the eye and creates a glint on the surface of the eye 20. The position of the glint relative to the eye-tracking device 12 is substantially stationary. Accordingly, as the user's eye and pupil moves to identify and track one or more items 23, such as provided on the display 16, the glint acts as a reference point for the camera 24.

In another example, the eye-tracking device 12 is a webcam-based eye tracking device. For example, the eye-tracking device 12 can be configured as a computerized device, such as a laptop, tablet, or mobile communication device having a controller, such as a processor and memory. The controller is disposed in electrical communication with a webcam and is configured to execute an eye-tracking application. During operation, the webcam can identify the location and orientation of the user's eyes 20 relative to the user's face and provide the location and orientation information to the controller. The controller is configured to map the eye location and orientation information to a coordinate system associated with a field of view, such as display 16, thereby allowing detection of the position of a user's eye relative to the display 16.

The health experience identification device 14 is configured as a computerized device, such as a personal computer, laptop, or tablet, and can include a controller 28, such as a processor and a memory. During operation, the health experience identification device 14 is configured to receive eye movement data 26 from the eye-tracking device 12 and to apply the eye movement data 26 to a health experience identification engine 40 to predict or identify a user's health experience, such as chronic pain or anxiety.

For example, as indicated above, those undergoing a health experience, such as pain, anxiety, or other experience that create an attentional bias in the person, differ in their cognitive processes, such as those involved in decision making, relative to those who are not undergoing that health experience. As such, the health experience can affect how those people process information and how they use that information to make decisions. Because eye-tracking provides unobtrusive insights into attention and cognition related to decision making, the health experience identification device 14 is configured to utilize the eye movement data 26 from the eye-tracking device 12 to differentiate the health experiences of people, such as between those with a health experience such as chronic pain and those with a different health experience, such as little to no pain.

As illustrated, during operation, the health experience identification device 14 is configured to apply the eye movement data 26 to a health experience identification engine 40, such as an artificial intelligence model, that is configured to output a health experience identifier 42 associated with the user's cognitive load as measured by the eye-tracking device 12.

The controller 28 of the health experience identification device 14 can store an application for identifying a user who is undergoing a health experience such as chronic pain or anxiety. The identification application installs on the controller 28 from a computer program product 30. In some arrangements, the computer program product 30 is available in a standard off-the-shelf form such as a shrink wrap package (e.g., CD-ROMs, diskettes, tapes, etc.). In other arrangements, the computer program product 30 is available in a different form, such downloadable online media. When performed on the controller 28 of the health experience identification device 14, the identification application causes the health experience identification device 14 to predict or identify a user's health experience based upon the eye movement data 26.

In one arrangement, the health experience identification device 14 is configured to generate the health experience identification engine 40. As indicated in FIG. 1, the health experience identification device 14 includes a health experience identification model 36, such as a neural network, deep learning, or tree type (e.g., random forest, decision, etc.) algorithm. The health experience identification device 14 can train the model 36 using health experience training data 38, such as historical user data, to create the health experience identification engine 40. This historical user data can be taken from multiple users or patients having previous interactions with the health experience identification system 10. While the health experience training data 38 can include any number of metrics, in one arrangement, the health experience training data 38 can include eye movement data 26, subjective health experience information, and objective health experience captured from the user.

During a training operation, the health experience identification device 14 can retrieve the health experience training data 38 from a database and apply the health experience training data 38 to the health experience identification model 36. With training of the health experience identification model 36, the health experience identification device 14 can develop the health experience identification engine 40. It is noted that the health experience identification device 14 can continuously train the health experience identification model 36 over time with additional health experience training data 38 (e.g., updated user data, new user data, etc.), such as retrieved from a database, to refine the health experience identification engine 40 over time.

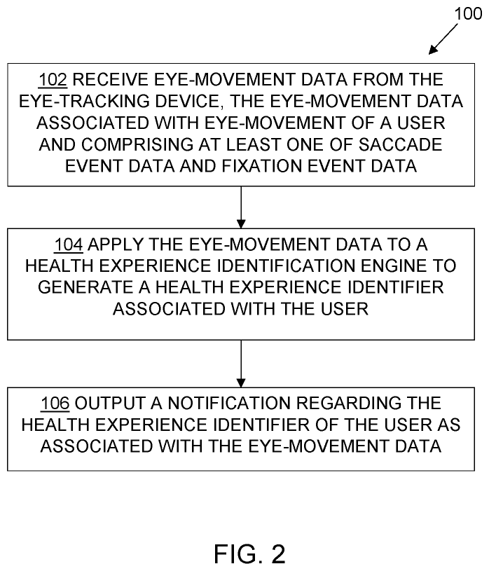

As provided above, during operation, the health experience detection device 14 is configured to predict the health experience of a user based upon the user's eye movements. FIG. 2 illustrates a flow chart 100 of a procedure performed by the health experience detection device 14 of the health experience identification system 10 of FIG. 1 when identifying a health experience of a user.

In element 102, the health experience detection device 14 is configured to receive eye-movement data 26 from the eye-tracking device 12, the eye-movement data 12 associated with eye-movement of a user and comprising at least one of saccade event data 29 and fixation event data 27.

For example, during operation and with reference to FIG. 1, as the user moves his eye 20 to identify and track various items 23 on the display 16, the eye-tracking device 12 is configured to detect the eye movements as related to either a fixation event or a saccade event. Fixation event data 27, identify fixations or pauses over informative regions of interest, along with the associated vertical and lateral coordinates (x, y). By contrast, saccade event data 29 identify relatively rapid movements, or saccades, between fixations used to recenter the eye on a new location, along with the vertical and lateral coordinate (x, y).

Further, it is noted that the eye-tracking device 12 can be configured to detect a pupil dilation event during the fixation or saccade event. For example, the eye-tracking device 12 can determine the diameter of the user's pupil or the rate of change of the user's pupil dilation as the pupil dilation event during either the fixation or saccade event. As a result of such eye motion detection, the eye-tracking device 12 can transmit eye-movement data 26 to the cognitive load detection device 14 that identifies the saccade event data 29 and the fixation event data 27, as well as pupil dilation event data.

Returning to FIG. 2, in element 104, the health experience detection device 14 is configured to apply the eye-movement data 26 to a health experience identification engine 40 to generate a health experience identifier 42 associated with the user. As provided above, the health experience identification engine 40 is configured to predict variety of types of user health experiences based upon the user's eye movements relative to the display 16.

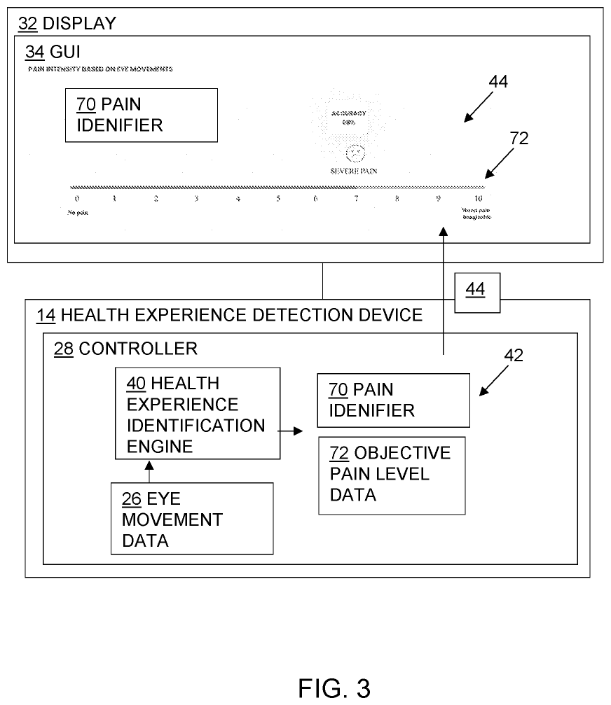

In one arrangement, with reference to FIG. 3, the health experience detection device 14 can apply the eye-movement data 26 to the health experience identification engine 40 to generate a pain identifier 70 and objective pain level data 72 associated with the user. For example, the pain identifier 70 can indicate a degree of pain experienced by the user, such as chronic pain or acute pain. Further, the objective pain level data 72 can identify an objective score associated with the pain experienced by the user. For example, the objective pain level data 72 can be a number on a scale of zero to ten where “zero” is indicative of no pain and “ten” is indicative of an extremely high level of pain.

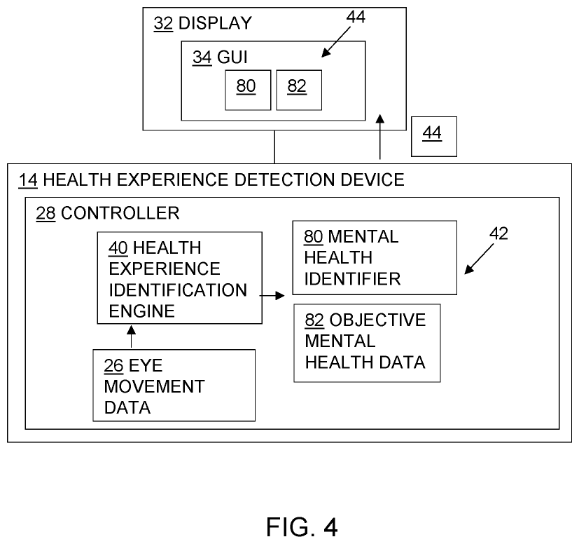

In one arrangement, with reference to FIG. 4, the health experience detection device 14 can apply the eye-movement data 26 to the health experience identification engine 40 to generate a mental health identifier 80 and objective mental health data 82 associated with the user. For example, the mental health identifier 80 can indicate a level of anxiety experienced by the user, such as chronic anxiety. Further, the objective mental health data 82 can identify an objective score associated with the anxiety experienced by the user. For example, the objective pain level data 72 can be a number on a scale of zero to ten where “zero” is indicative of no anxiety and “ten” is indicative of high anxiety.

Returning to FIG. 2, in element 106, the health experience detection device 14 is configured to output a notification 44 regarding the health experience identifier 42 of the user as associated with the eye-movement data 26. In one arrangement, with reference to FIG. 1, the health experience detection device 14 can output a notification 44 to a graphical user interface (GUI) provided by the display 16. With such display, the notification 44 can provide information regarding the user's predicted or detected health experience to a healthcare professional. For example, the notification can indicate that the user is experiencing a chronic pain condition.

In one arrangement, with reference to FIG. 3, when outputting the notification 44 regarding the health experience identifier 42, the health experience detection device 14 can be configured to display the pain identifier 70 and the objective pain level data 72 associated with the user. For example, the pain identifier 70 can indicate the user's pain as either being chronic or acute. Additionally, the objective pain level data 72 can identify a pain intensity score, such as an objective score on a scale of one to ten, along with pain qualifier text related to the score, such as “low pain” or “severe pain.”

In one arrangement, with reference to FIG. 4 when outputting the notification 44 regarding the health experience identifier 42, the health experience detection device 14 can be configured to display the mental health identifier 80 and the objective mental health data 82 associated with the user. For example, the mental health identifier 80 can indicate the user as having anxiety. Additionally, the objective mental health data 82 can identify a mental health intensity score, such as an objective score on a scale of one to ten, along with anxiety qualifier text related to the score, such as “low anxiety” or “severe anxiety.”

The health experience detection system 10 is configured to track a user's eye movements and to predict a health experience associated with the user via a health experience identification engine 40. As provided above, it is known that a health experience, such as chronic pain or anxiety, can interrupt cognition and create an attentional bias in a person. As such, the health experience detection device 14 executing the health experience identification engine 40 can accurately and objectively predict the user's health experience via the eye-movement data 26. By providing an objective measure of the user's health experience (e.g., chronic vs. acute pain, chronic vs. acute anxiety, etc.), the health experience detection device 14 provides a health care professional with the information needed to accurately assess and treat the user's health condition.

As provided above, to generate the eye-movement data 26, the user can visually track one or more items 23, such as provided on the display 16. In one arrangement, and with reference to FIG. 4, the health experience detection device 14 is configured to present a visual stimuli sample 55 related to a health experience topic associated with the user via the display 16.

For example, when a user engages the health experience identification system 10, the health experience detection device 14 can retrieve health experience information 57 associated with the user, either from a health experience database 56 or as provided by the user. Based upon the health experience information 57, the health experience detection device 14 present a visual stimuli sample 55 related to the health experience information 57.

For example, assume the case where the user believes himself to have chronic back pain. The user can provide health experience information 57 to the health experience detection device 14, such as via a survey, of the occurrence of the back pain. As a result, the health experience detection device 14 can present as a visual stimuli sample 55 on the display 16 which relates to back pain. As the user views the visual stimuli sample 55 the health experience detection device 14 receives associated eye-movement data 26 from the eye-tracking device 12. With the visual stimuli sample 55 being related to back pain, as the user reviews the sample 55, the sample 55 can trigger an attentional bias related to the user's own pain. Such attentional bias can be tracked by the eye-tracking device 12 and can be identified by the health experience detection device 14. As such, by presenting the visual stimuli sample 55 related to the user's health experience information 57, the health experience detection device 14 can increase the accuracy of the health experience identifier 42 generated by the health experience identification engine 40.

The visual stimuli sample 55 can be configured in a variety of ways. For example, the visual stimuli sample 55 can be an image or text related to the user's health experience information 57, such as a picture or paragraph related to back pain. The visual stimuli sample 55 can also be a subjective survey, such as a survey related to the pain of the user but not specifically about the pain itself. For example, the survey can include questions such as “Does pain interfere with daily routine?” and “How difficult is it for you to go up and down stairs?”.

In one arrangement, in order to mitigate the presence of subjective bias from influencing the eye-movement data 26, the health experience detection device 14 can be configured to provide a customized visual stimuli sample 55 to the user based upon the user's health experience information 57. Such provision can be done in a variety of ways.

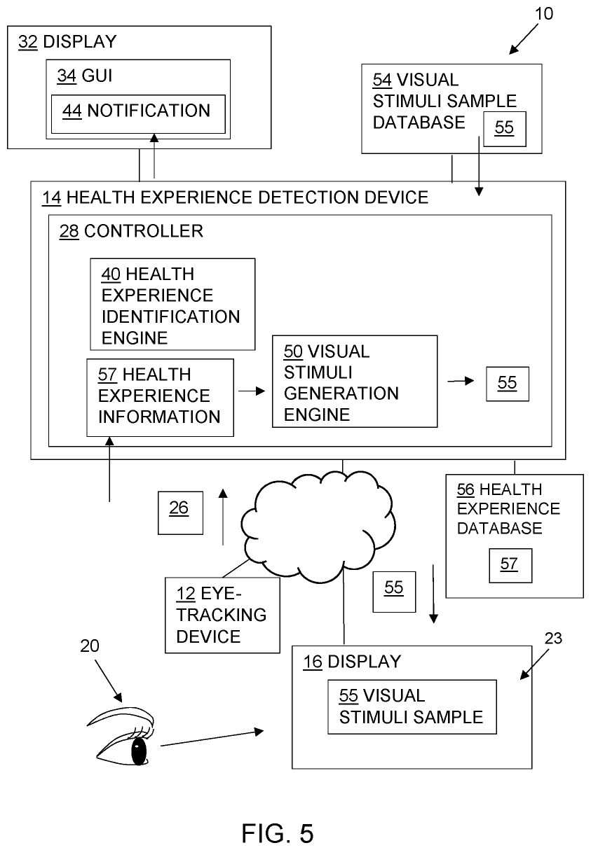

For example, as shown in FIG. 5, the health experience detection device 14 can select a visual stimuli sample 55 from a visual stimuli sample database 50 based upon health experience information 57 associated with the user. As provided above, assume the case where the user believes himself to have chronic back pain and the user provides health experience information 57 to the health experience detection device 14 indicating the occurrence of the back pain. Based upon the health experience information 57, the health experience detection device 14 can select, as the visual stimuli sample 55, either a paragraph describing or an image showing, a person experiencing back pain and present that custom selected visual stimuli sample 55 to the user via the display 16.

In another example, as also shown in FIG. 5, the health experience detection device 14 can be configured to generate the visual stimuli sample 55 based upon the health experience information 57 associated with the user. Assume the case where the user has developed historical health experience information 57 with a facility associated with the health experience detection device 14 and which is stored within the health experience database 56. When the user arrives at the facility and the health experience detection device 14 receives notification of the user's arrival, the health experience detection device 14 can access the health experience database 56 to retrieve the historical health experience information 57 related to that user (e.g., showing the user has chronic back pain). The health experience detection device 14 can then apply the historical health experience information 57 to a visual stimuli generation engine 50, such as generated through the training of an artificial intelligence model. As a result of applying the historical health experience information 57 to the visual stimuli generation engine 50, the visual stimuli generation engine 50 can generate a visual stimuli sample 55 specific to the user and related to the user's historical health experience information 57.

Returning to FIG. 1 and as indicated above, the health experience detection device 14 can be configured to train a health experience identification model 36 based on historical user data taken from multiple users to create the health experience identification engine 40. In one arrangement, to mitigate the presence of bias within the health experience identification engine 40, the health experience detection device 14 can be configured to train the health experience identification model 36 with training data specific to a particular user to generate a user-specific health experience identification engine 60.

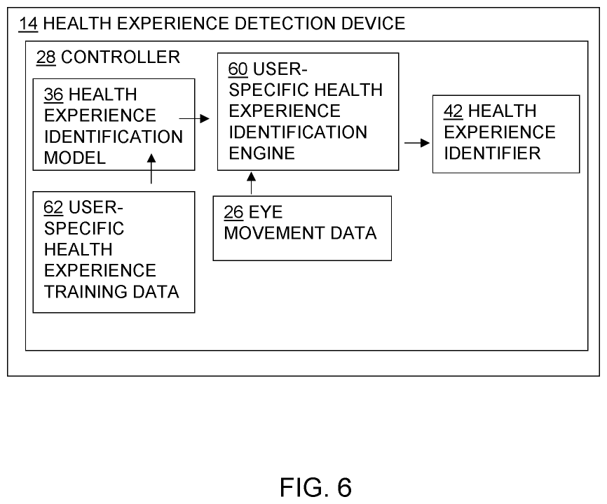

For example, with reference to FIG. 6, assume the case where the health experience detection device 14 receives notification that a particular user will be interacting with the system 10. In such a case, the health experience detection device 14 can retrieve user-specific health experience training data 62 associated with that user. For example, the device 14 can access a database, such as the health experience database 56, and can retrieve health experience information 57 pertaining to that user. The health experience detection device 14 can then apply that health experience information 57 as user-specific health experience training data 62 to the health experience identification model 36. Such application trains the model 36 and results in a user-specific health experience identification engine 60.

During operation, when the health experience detection device 14 receives eye-movement data 26 from that user, the device 14 can apply the eye-movement data 26 to the user-specific health experience identification engine 60 to generate the health experience identifier 42 associated with the user. With use of the engine, 60 the health experience detection device 14 and can more accurately predict the health experience identifier 42 associated with the user, thereby leading to an accurate diagnosis by a healthcare professional.

Returning to FIG. 1, as provided above the healthcare expert experience detection device 14 is configure to generate a notification 44 based upon the health experience identifier 42 generated by the health experience identification engine 40 and which provides information regarding the user's predicted or detected health experience. In one arrangement, the healthcare expert experience detection device 14 is further configured to provide a treatment recommendation 48 for the user based upon the health experience identifier 42.

For example, following generation of the health experience identifier 42 by the health experience identification engine 40, the health detection device 14 can apply the health experience identifier 42 to a diagnosis engine 46. In one arrangement, the health detection device 14 has developed the diagnosis engine 46, such as through the training of an artificial intelligence model, to generate one or more treat treatment recommendations 48 based upon a health experience identifier 42. For example, assume the case where the health experience identifier 42 identifies the user as having chronic back pain. Application of such a health identifier 42 to the diagnosis engine 46 can result in a treatment recommendation 48 being generated which identifies treatments that can provide relief of the chronic back pain. For example, the treatment recommendation 48 can identify a particular medication or exercise regimens to mitigate or alleviate the user's chronic back pain.

Following generation of the treatment recommendation 48, the health detection device 14 can output the treatment recommendation 48, as associated with the health experience identifier 42. For example, the health detection device 14 can transmit the treatment recommendation 48 to the display 32 to be presented to a healthcare worker as part of the GUI 34. The healthcare worker can then utilize the treatment recommendation 48 as part of a care plan for the user.

While various embodiments of the innovation have been particularly shown and described, it will be understood by those skilled in the art that various changes in form and details may be made therein without departing from the spirit and scope of the innovation as defined by the appended claims.

What is claimed is:

1. A health experience detection system, comprising:
an eye-tracking device; and
a health experience detection device disposed in electrical communication with the eye-tracking device, the health experience detection device comprising a controller having a memory and a processor, the controller configured to:
receive eye-movement data from the eye-tracking device, the eye-movement data associated with eye-movement of a user and comprising at least one of saccade event data and fixation event data,
apply the eye-movement data to a health experience identification engine to generate a health experience identifier associated with the user, and
output a notification regarding the health experience identifier of the user as associated with the eye-movement data.

2. The health experience detection system of claim 1, wherein:
when receiving eye-movement data from the eye-tracking device, the controller is configured to:
present a visual stimuli sample related to a health experience topic to the user via a display; and
receive eye-movement data from the eye-tracking device, the eye-movement data associated with viewing of the visual stimuli sample by the user.

3. The health experience detection system of claim 2, wherein the controller is further configured to select the visual stimuli sample from a visual stimuli sample stimuli database, the visual stimuli sample related to health experience information associated with the user.

4. The health experience detection system of claim 2, wherein the controller is further configured to generate the visual stimuli sample based upon a health experience information associated with the user.

5. The health experience detection system of claim 1, wherein when applying the eye-movement data to the health experience identification engine to generate the health experience identifier associated with the user, the controller is configured to apply the eye-movement data to a user-specific health experience identification engine to generate the health experience identifier associated with the user.

6. The health experience detection system of claim 1, wherein:
when applying the eye-movement data to the health experience identification engine to generate the health experience identifier associated with the user, the controller is configured to apply the eye-movement data to the health experience identification engine to generate a pain identifier and objective pain level data associated with the user; and
when outputting the notification regarding the health experience identifier of the user as associated with the eye-movement data, the controller is configured to display the pain identifier and the objective pain level data associated with the user.

7. The health experience detection system of claim 1, wherein:
when applying the eye-movement data to the health experience identification engine to generate the health experience identifier associated with the user, the controller is configured to apply the eye-movement data to the health experience identification engine to generate a mental health identifier and objective mental health data associated with the user; and
when outputting the notification regarding the health experience identifier of the user as associated with the eye-movement data, the controller is configured to display the mental health identifier and the objective mental health data associated with the user.

8. The health experience detection system of claim 1, wherein the controller is further configured to:
apply the health experience identifier to a diagnosis engine to generate a treatment recommendation associated with the user; and
output the treatment recommendation as associated with the health experience identifier.

9. In a health experience detection device, a method for providing a health experience identifier of a user, comprising:
receiving, by the health experience detection device, eye-movement data from an eye-tracking device, the eye-movement data associated with eye-movement of the user and comprising at least one of saccade event data and fixation event data;
applying, by the health experience detection device, the eye-movement data to a health experience identification engine to generate a health experience identifier associated with the user; and
outputting, by the health experience detection device, a notification regarding the health experience identifier of the user as associated with the eye-movement data.

10. The method of claim 9, wherein:
receiving eye-movement data from the eye-tracking device comprises:
presenting, by the health experience detection device, a visual stimuli sample related to a health experience topic to the user via a display, and
receiving, by the health experience detection device, eye-movement data from the eye-tracking device, the eye-movement data associated with viewing of the visual stimuli sample by the user.

11. The method of claim 10, further comprising selecting, by the health experience detection device, the visual stimuli sample from a visual stimuli sample stimuli database, the visual stimuli sample related to a health experience information associated with the user.

12. The method of claim 10, further comprising generating, by the health experience detection device, the visual stimuli sample based upon a health experience information associated with the user.

13. The method of claim 9, wherein applying the eye-movement data to the health experience identification engine to generate the health experience identifier associated with the user comprises applying, by the health experience detection device, the eye-movement data to a user-specific health experience identification engine to generate the health experience identifier associated with the user.

14. The method of claim 9, wherein:
applying the eye-movement data to the health experience identification engine to generate the health experience identifier associated with the user comprises applying, by the health experience detection device, the eye-movement data to the health experience identification engine to generate a pain identifier and objective pain level data associated with the user; and
outputting the notification regarding the health experience identifier of the user as associated with the eye-movement data comprises displaying, by the health experience detection device, the pain identifier and the objective pain level data associated with the user.

15. The method of claim 9, wherein:
applying the eye-movement data to the health experience identification engine to generate the health experience identifier associated with the user comprises applying, by the health experience detection device, the eye-movement data to the health experience identification engine to generate a mental health identifier and objective mental health data associated with the user; and
outputting the notification regarding the health experience identifier of the user as associated with the eye-movement data comprises displaying, by the health experience detection device, the mental health identifier and the objective mental health data associated with the user.

16. The method of claim 9, further comprising:
applying, by the health experience detection device, the health experience identifier to a diagnosis engine to generate a treatment recommendation associated with the user; and
outputting, by the health experience detection device, the treatment recommendation as associated with the health experience identifier.

17. A health experience detection device, comprising:
a controller having a memory and a processor, the controller configured to:
receive eye-movement data from an eye-tracking device, the eye-movement data associated with eye-movement of a user and comprising at least one of saccade event data and fixation event data;
apply the eye-movement data to a health experience identification engine to generate a health experience identifier associated with the user; and
output a notification regarding the health experience identifier of the user as associated with the eye-movement data.
